(19) United States
(12) Patent Application Publication
(10) Pub. No.: US 2023/0320886 A1
(43) Pub. Date: Oct. 12, 2023

(54) Convertible Lumbar Sacral Orthosis Support Device

(71) Applicant: Donald Francisco, SAN JUAN CAPO, CA (US)

(72) Inventor: Donald Francisco, SAN JUAN CAPO, CA (US)

(21) Appl. No.: 18/059,876

(22) Filed: Nov. 29, 2022

Related U.S. Application Data
(60) Provisional application No. 63/323,605, filed on Mar. 25, 2022.

(51) Int. Cl. A61F 5/02; A61F 7/02

(52) U.S. Cl. CPC A61F 5/028; A61F 7/02; A61F 2007/0219; A61F 2007/023; A61F 2007/0285

(57) ABSTRACT

The present invention provides a support band that comprises a size adjustable flexible abdominal belt including a back panel, a right side contouring panel, a left side contouring panel, a right front end panel, and a left front end panel, an internal pocket, an external pocket, one or more internal sleeve pockets, a plurality of compression straps, and a plurality of anterior rigid panels, and a plurality of posterior rigid panels.

### FIELD OF THE INVENTION

The present invention relates generally to a lumbar sacral orthosis support device. More specifically, the present invention is a convertible orthotic support device that can be personally configured by the user between flexible, semi-rigid, rigid, and therapeutic levels of designated support within a single device by utilizing a circumferential support band structure, a removable posterior rigid panel, a removable anterior rigid panel, detachable compression straps,, and a removable therapeutic gel pack.

### BACKGROUND OF THE INVENTION

A typical lumbar sacral orthosis device is a type of orthotic support band worn around the waist and lower back for multiple purposes such as improved posture and core stability, lower back pain relief, and reduced spinal cord compression stress. Products in the marketplace are specifically and historically designed, marketed, and subject to a single level of support and functionality, typically flexible or rigid orthotic designations without the ability to be adjusted, or configured to another support designation, offering convertibility to the user, rendering the need for multiple products to achieve a range of support needs and indications for use.

An objective of the present invention is to provide an improved lumbar sacral orthosis device that is convertible, versatile, multi-functional, and personally customizable, permitting users to convert between the four designated levels of support (flexible, semi-rigid, rigid, and therapeutic) with a single device through the application of removable rigid support panels in the anterior and posterior of the device, detachable compression straps, and therapeutic heat/ice features. To accommodate a broad range of user needs and applications, the unique rigid support panels are easily inserted and/or removed from the device by way of accompanying interior or exterior pockets or sleeves, allowing for an instant on-demand convertible range of support without the need for additional devices to achieve. In addition, supplemental design features allow further customization by the user to customize the desired level of compression support with the use of detachable compression straps permitting a user to adjust support levels in a variety of support configurations, on demand and with repeatability consistency. The present invention can be worn in eight different configurations to capture a comprehensive set of users’ physiological support and/or therapeutic needs, while maintaining the unique convertibility between flexible and rigid support designations as defined by orthotic medical devices and durable medical equipment.

No other orthotic device incorporates or is comparable to the versatile convertibility of the present invention through unique design features, functionality, construction, and application for the widest range of support, giving the user full control over how the device is configured for a personal level of support. The present invention is designed with the utility of interchangeable functions and provides the broadest range of support, structure, and customized fit for individual use case scenarios. Furthermore, these unique features are specifically designed to be easily converted, added, or removed as to not be permanently affixed to the device, providing further versatility in the device configuration and user experience. Similar products on the market are designed with permanent features that are fixed to the device and do not allow conversions and/or configuring of any kind, requiring multiple versions of similar devices to achieve this range of support, use, and application. Additional features and benefits are further discussed in the sections below.

### BRIEF SUMMARY

This summary is provided to introduce a selection of concepts in a simplified form, that are further described below in the Detailed Description. This summary is not intended to identify key features or essential features of the claimed subject matter. Nor is this summary intended to be used to limit the claimed subject matter’s scope.

The present invention provides a support band that comprises a size adjustable convertible abdominal band including a back panel, a right side contouring panel, a left side contouring panel, a right front end panel, and a left front end panel, an internal pocket, an external pocket, one or more internal sleeve pockets, a plurality of compression straps, and a plurality of anterior rigid panels, and a plurality of posterior rigid panels.

In one embodiment, the compression strap may include a plurality of detachable compression straps having one or more fasteners at one or more ends of the compression straps.

In some embodiments, the present invention may further include a therapeutic gel pack that is a therapeutic heating and cooling accessory. The therapeutic gel pack can be inserted into interior pockets of the present invention to provide pain relief, reduce swelling, and soothing therapeutic benefit to the user when indicated and/or desired.

### BRIEF DESCRIPTION OF THE DRAWINGS

FIG. 1 is a front view showing an internal pocket, an internal sleeve pocket, a back panel, a right side contouring panel, a left side contouring panel, a right front end panel, and a left front end panel of the present invention.

FIG. 2 is a rear view showing an external pocket, an external sleeve pocket, a back panel, a right side contouring panel, a left side contouring panel, a right front end panel, and a left front end panel of the present invention.

FIG. 3 is an illustration showing a compression strap of the present invention.

FIG. 4 is an illustration showing how the support band and the compression straps are attached.

FIG. 5 is an illustration showing how the anterior rigid panels and the therapeutic gel pack are inserted into the internal pocket and the internal sleeve pocket of the present invention.

FIG. 6 is an illustration showing how posterior rigid panel is inserted into the external pocket of the present invention without the external sleeve pocket.

### DETAIL DESCRIPTIONS OF THE INVENTION

All illustrations of the drawings are for the purpose of describing selected versions of the present invention and are not intended to limit the scope of the present invention.

The present invention provides a convertible lumbar sacral orthosis support device. The present invention is configured as convertible, versatile, multi-functional, and personally customizable, permitting users to convert between the four designated levels of support with a single device through the application of removable rigid panels in the anterior and posterior of the device, detachable compression straps, and therapeutic heat/ice features.

The four designated levels of support are flexible, semi-rigid, rigid., and therapeutic. The flexible level support is configured for mild support needs and flexibility. For example, the flexible level support configuration does not include or use the anterior or posterior rigid panels, but detachable compression straps may be used in this configuration depending on the desired level of compression in the flexible configuration. The semi-rigid level support is configured for moderate support needs, where firmer support is indicated while maintaining flexibility.

The semi-rigid level support is established by combining the convertible features. For example, the detachable compression straps are attached and applied to adjust compression levels. While the front portion of the present invention remains flexible, the posterior rigid panel is inserted to provide a rigid level of support in the back of the present invention.

The rigid level support is configured for firm support needs, where rigid and strong stability is the priority. For example. the detachable compression straps are attached and applied to maintain the rigid level of support in chorus with the use of the anterior and posterior rigid panels.

The therapeutic level support is configured for healing or therapy needs. For example, a therapeutic gel pack is inserted into the internal pocket for use as Heat or Ice therapy and the detachable compression straps may be used in this configuration depending on the desired level of compression to assist in applying heat or ice. The rigid panels are not used in this configuration when solely seeking therapeutic benefits without designated orthotic support.

The present invention is comprised and assembled of performance materials, fabrics, and trims including, Lycra, Nylon, Air-Mesh, Elastic, Loop Fabric, Polyethylene, but may also include additional performance fabric compositions and/or additional composite materials. The device is applied around a person’s body in a circumferential formation, attaching in the front with Velcro or other fastener trims.

The present invention provides a circumferential band structure that can wrap around the user’s waist, supporting the lower back, sacrum, hips, waist, and abdominal areas. In one embodiment, the present invention provides a size adjustable flexible abdominal belt (support band) 100 with elongated ends (a first and second ends, 10, 11).

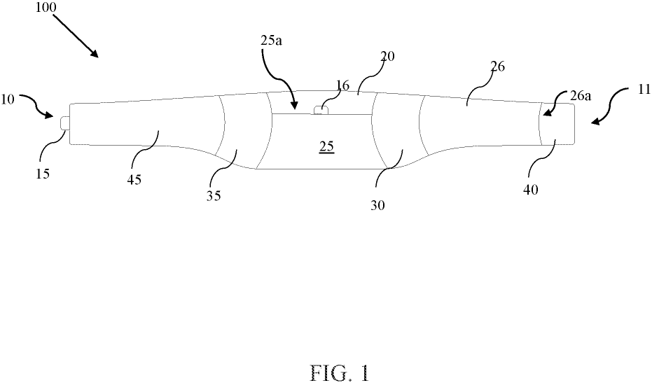

In the preferred embodiment, as shown in FIG. 1, the present invention (support band 100) comprises five functional panels and is constructed together with a sewn seam, bonded, or attached by other means of adhesion to form a circular support device that is applied around the body.

As shown in FIGS. 1- 6, the five functional panels of the present invention include a back panel 20, a right side contouring panel 30, a left side contouring panel 35, a right front end panel 40, and a left front end panel 45. The back panel 20 can be made from Air-Mesh, EVA Foam, Neoprene, or other contouring and/or flexible materials.

The back panel 20 can be attached to the side contouring panels 30, 35 via sewn seams, bonding, or other assembly methods. Further, in some embodiments, the back panel 20 can be bonded on the inside layer with performance fabrics like Nylon/Lycra or cotton compositions where in direct contact with skin.

The right side contouring panel 30 can be attached to the right side of the back panel 20 and right side of the front portion of the present invention, to complete the right side of the size adjustable flexible abdominal belt (support band) 100, and the attachments can be by sewn seams, bonding, or other adhesion technique. The right side contouring panel 30 can be made from performance fabrics like Nylon, Lycra, EVA Foam, or Neoprene materials.

The left side contouring panel 35 can be attached to the left side of the back panel 20 and the left side of the front portion of the present invention, to complete the left side of the size adjustable flexible abdominal belt (support band) 100, and the attachments can be by sewn seams, bonding, or other adhesion technique.

The left side contouring panel 35 can also be made from performance fabrics like Nylon, Lycra, EVA Foam, or Neoprene materials.

The right front end panel 40 can be attached to the right side contouring panels 30 via sewn seams, bonding, or other methods of assembly.

The right front end panel 40 made from Air-Mesh, Nylon, EVA foam, Neoprene, or other flexible materials.

In some embodiments, the right front end panel 40 can be bonded or laminated with a loop style fabric on the back side to accept hook or Velcro materials. Further, in some embodiments, the right front end panel is bonded with performance fabrics Nylon/Spandex or Cotton blends on the front side where in direct contact with the skin of the user.

The left front end panel 45 can be attached to the left side contouring panels 35 via sewn seams, bonding, or other methods of assembly. Similar to the right front end panel 40, the left front end panel 45 can be made from Air-Mesh, Nylon, EVA foam, Neoprene, or other flexible materials.

In some embodiments, the left front end panel 45 can also be bonded or laminated with a loop style fabric on the back side to accept hook or Velcro materials. Further, in some embodiments, the left front end panel 45 can also be bonded with performance fabrics Nylon/Spandex or Cotton blends on the front side where in direct contact with the skin.

In some embodiments, the size adjustable flexible abdominal belt (support band) 100 may include a connector 15 for connecting a first end (which may be one end of the left front end panel 45) and a second end (which may be one end of the right front end panel 40) of the size adjustable flexible abdominal belt.

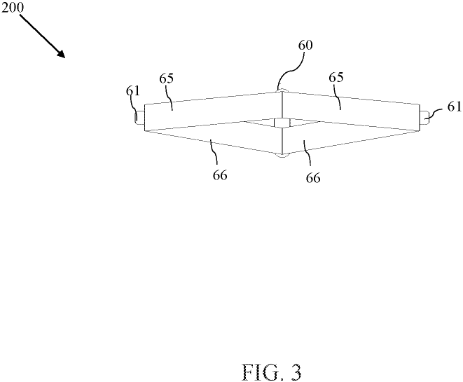

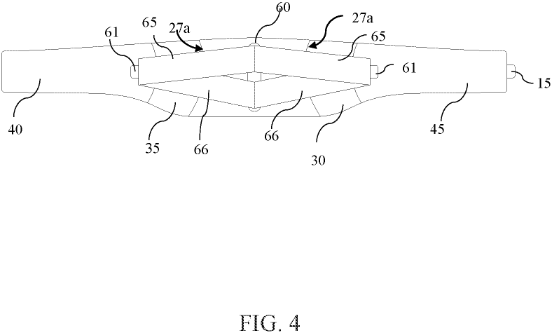

Furthermore, as shown in FIGS. 3-4, the support band (the size adjustable flexible abdominal belt 100) may further comprise a strap support 60 a compression strap 200 that provide adjustable compression and is made from medical grade elastic material and may vary in composition depending on the application.

In one embodiment, the compression strap 200 may include a plurality of detachable compression straps 200 having one or more fasteners 61 at one or more ends of the compression straps 200. Such compression strap 200 may include multiple elastic straps 65, 66.

In some embodiments, the elastic straps 65, 66 can include at least one elongated element and means to attach ends of the elastic straps together. In some embodiments, the compression strap 200 may include one or more upper elastic strap 65 and lower elastic straps 66. The ends of the elastic straps (upper elastic strap and a lower elastic strap 65, 66) can be attached together, and the elastic straps 65, 66 can be attached to the strap support 60 to form an elastic diamond shape.

In some embodiments, the elastic strap 65, 66 may comprise one or more straps of pre-determined lengths and elastic straps 65, 66 can be selected based on the length needed for a user.

The compression strap 200 can be applied from a back of a user to front direction, stretching into place and attaching in the front with Velcro or other fastener trims to achieve a desired level of compression. The compression straps 200 can be secured in place in the back of the size adjustable flexible abdominal belt (support band) 100 with detachable Velcro or other quick detachable fasteners.

The aforementioned contouring side panels 30, 35 can be flexible and may allow anatomical contouring to ensure a personal fit that is easily adjusted to accommodate a wide variety of body types, sizes, and shapes, ensuring an accurate and effective contouring fit, without binding, folding, or otherwise becoming deformed and compromising fit and function of the entire device (support band 100) of the present invention. These contouring side panels 30, 35 can be fabric panels made from a Nylon/Lycra composition and may also include other performance fabrics.

The compression strap 200, which can be detachable from the size adjustable flexible abdominal belt (support band) 100 can include memory retention medical grade elastic straps that provide the user the ability to personally adjust and temper the amount of compression applied to the device, pulling into place from back to a front orientation on both the right and left side of the support band 100, attaching in the center back of the support band 100.

The versatility of this compression component (compression strap 200) can also be completely removed by easily detaching from the support band 100 to convert the support band 100 between designated levels of support, namely changing from rigid, or semi-rigid levels of support, down to a flexible level of support, and also a therapeutic level when a heat or ice gel pack 80 is applied and in use.

In some embodiments, the compression strap 200 can be easily attached and detached in the center back of the support band 100 with a Velcro attachment system that secures the compression straps in place 200. When stretched into position, the compression strap 200 can be secured in place on the front portion of the user with Velcro and a loop style fabric, allowing for repositioning and repeated use.

The detachable compression strap 200 can be made of medical grade elastic composition constructed from Nylon, Elastic, Polyester, and other stretch materials secured in position with detachable Velcro or other shear fasteners to be converted to the desired use.

The detachable compression strap 200 can be stretched into position with “finger pulls” made of urethane or other flexible rubber fastened or sewn on the ends of the elastic straps 65, 66 to assist in pulling the elastic straps 65, 66 into place. In some other embodiments, the detachable compression strap 200 can also be secured to the front portion of the support band 100 with Velcro and/or hook and loop materials sewn or bonded to the underside of the ends of the elastic straps 65, 66 and attached to the front of the support band 100 via loop fabric. In the preferred embodiment, the detachable compression strap 200 may provide dimensions ranging from 65-80 mm in width and 20-45 cm in length.

In some embodiments, the present invention may further include one or more anterior rigid panels 91 and posterior rigid panels 92 that are custom molded and anatomically correct rigid panels that provide a semi-rigid (moderate) and/or rigid (firm) levels of support.

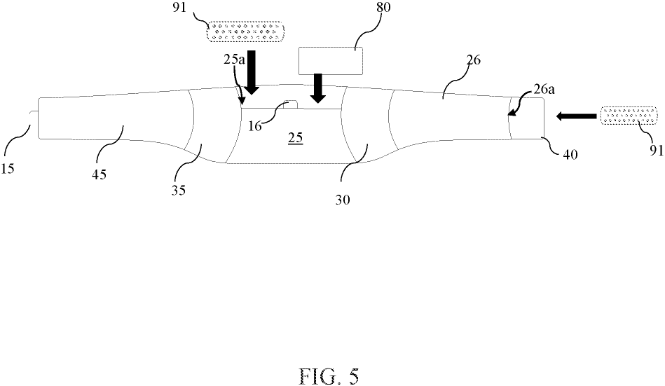

As shown in FIG. 5, the anterior rigid panels 91 can be inserted into an internal pocket or sleeve 26 (having a first opening 26a) which may be positioned in the center of front side of the support band 100.

In some embodiments, the internal pocket 26 can be disposed in a mid-portion of a front side of the size adjustable flexible abdominal belt 100, the internal pocket 26 having a first opening for accessing the internal pocket 26a.

The anterior rigid panels 91 can also be inserted into one or more internal sleeve pockets 26 (having a third opening 26a) which may be positioned on the front side of the left or right front end panel 40, 45.

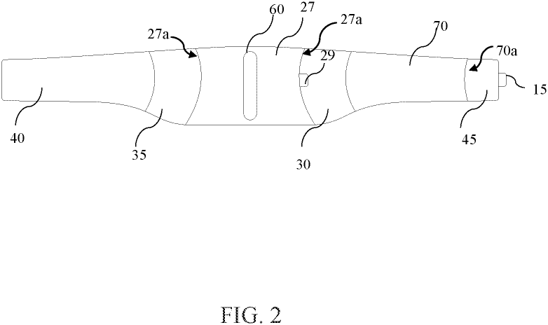

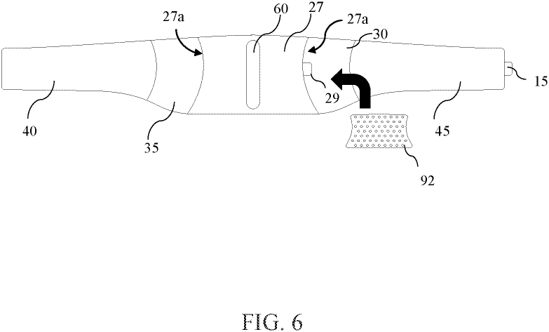

As shown in FIG. 6, in some alternative embodiments, the present invention may not include the exterior sleeve pocket 27 shown in FIG. 2.

As shown in FIG. 6, the posterior rigid panels 92 can be inserted to an external pocket 27 which may be positioned on the center of the back side of the support band 100, providing a firm or rigid level of support to the target areas indicated for treatment.

In some embodiments, the external pocket 27 can be disposed in a mid-portion of a back side of the size adjustable flexible abdominal belt 100, the external pocket 27 having a second opening 27a for accessing the external pocket 27.

The posterior rigid panels 92 can also be inserted into one or more external sleeve pockets 70 (having a fourth opening 70a) which may be positioned on the back side of the left or right front end panel 40, 45.

In the preferred embodiment, the rigid panels 91, 92 provide anatomical shaped structures with a plurality of holes and/or perforations permitting air flow that fits the corresponding pockets 25, 26, 27. The rigid panels 91, 92 can be made from a form-fitting composite plastic and custom molded to be anatomically correct and retain shape and integrity throughout extended use.

When compressed against the user’s body, natural body heat assists in molding the panel for a contouring fit to enhance function and improve comfort. The composite plastic material can be lightweight, durable, and molded with a unique air-hole and/or a perforated pattern that permits airflow for performance, while maintaining a rigid structure to support a firm or semi-firm support level designation. These rigid panels 91, 92 can be easily inserted and removed from the pockets 25, 26, 27 that can be placed any suitable places including the internal and/or the external sides of the support band 100 and can be used interchangeably to covert between rigid, semi-rigid, and flexible levels of support designation. In some embodiments, the rigid panels 91, 92 can be ergonomically and anatomically shaped to contour the targeted area being treated to form a contouring fit for effective structural support.

In the preferred embodiment, the anterior rigid panel 91 is inserted into (or detachably/slidably attached in) internal sleeve pocket 26. The anterior rigid panel 91 may be laterally adjusted to the center of the targeted body area.

In some embodiments, the internal sleeve pocket 26 can be hidden inside the front portion of the support band 100 having end openings and accessible from both ends allowing the panel 91 to be positioned freely inside the internal sleeve pocket 26 when the present invention is flat open before the present invention is closed.

When the rigid panels 91, 92 are not inserted, the present invention is converted to a flexible level of support for users indicated for mild to moderate levels or orthotic support.

In some embodiments, the posterior rigid panel 92 can be inserted into an external pocket 27 in a fixed position providing a semi-rigid, or rigid level of support. In some embodiments, the external pocket 27 can be hidden under the detachable compression straps 200 and accessible by an opening (second opening 27a) on the right hand side and secured closed with a Velcro tab (or similar fastener) 29. When the posterior rigid panel 92 is not inserted, the present invention is converted to a flexible level of support for users indicated for mild levels of orthotic support.

In the preferred embodiment, the pockets 25, 26, 27 are made of Performance fabric such as Lycra/Nylon blend composition or other material like neoprene to provide strength and a secure pocket while laying flat and contour the body area holding the rigid panels in place without being cumbersome and allowing for air-flow to keep the device (support band 100) cool and comfortable. The pockets 25, 26, 27 may include a side or top opening and can be securely closed with Velcro or other shear fasteners.

The present invention may further include a therapeutic gel pack 80 that is a therapeutic heating and cooling accessory. The therapeutic gel pack 80 can be inserted into any pockets 25, 26, 27 to provide pain relief, reduce swelling, and soothing therapeutic benefit to the user when indicated and/or desired. The therapeutic gel pack can be inserted in (or detachably/slidably attached in) any pockets 25, 26, 27.

The therapeutic gel pack 80 may be used as heat therapy by applying microwave heat or placing it in warm water to the desired temperature. The therapeutic gel pack 80 may also be used as ice or cold therapy by placing in the freezer until the gel is cold or frozen. The therapeutic gel pack 80 can be easily inserted and removed without altering the present invention, adding further convertibility and functionality to a therapeutic level of support when flexible and/or rigid levels of support are not indicated or desired, and also adding therapeutic functionally to the designated levels of support, flexible, semi-rigid, and rigid, which in turn upgrades these three levels of support to include therapy in addition to orthotic support, expanding the versatility and functionality of the present invention. The therapeutic gel pack contents can be water-based gel and encapsulated in a Nylon or other performance material composite fabric shell.

Based on the user’s preference, the present invention can be worn in eight different configurations to capture a comprehensive set of support designations and personal fit. The first configuration is the use of the support band 100 only. The second configuration is the support band 100 and detachable compression straps 200 combination. The third configuration is the support band 100, detachable compression straps 200, and anterior rigid panel 91 combination.

The fourth configuration is the support band 100, detachable compression straps 200, and posterior rigid panel 92 combination. The fifth configuration is the support band 100, detachable compression straps 200, anterior rigid panel 91, and posterior rigid panel 92 combination.

The sixth configuration is the support band 100, detachable compression straps 200, anterior rigid panel 91, posterior rigid panel 92, and therapeutic gel pack 80 combination. The seventh configuration is the support band 100, detachable compression straps 200, and therapeutic gel pack 80 combination. The eighth configuration is the support band 100 and therapeutic gel pack 80 combinations.

In the preferred embodiment, the present invention provides 10″ of length adjustability in the support band 100 of the present invention to accommodate a variety of body sizes, shapes, and levels of comfort. The support band 100 is attached and repositioned with ease using Velcro and loop fabric bonded to the front end panels 40, 45 of the support of the present invention.

The present invention will close securely in the front with Velcro, hook and/or loop, or other shear fasteners on each end of the support band, creating a secure closure that allows for length adjustments and repositioning.

Although the invention has been explained in relation to its preferred embodiment, it is to be understood that many other possible modifications and variations can be made without departing from the spirit and scope of the invention.

What is claimed is:

1. A device comprising,
a size adjustable flexible abdominal lower lumbar band including:
a back panel,
a right side contouring panel attached to the right side of the back panel,
a left side contouring panel attached to the left side of the back panel,
a right front end panel attached to one end of the right side contouring panel, and
a left front end panel attached to one end of the left side contouring panel;
a connector for connecting a first end and a second end of the size adjustable flexible abdominal belt;
an internal pocket disposed in a mid-portion of a front side of the size adjustable flexible abdominal belt, the internal pocket having a first opening for accessing the internal pocket for a panel or pack;
an external pocket disposed in a mid-portion of a back side of the size adjustable flexible abdominal belt, the external pocket having a second opening for accessing the external pocket for a panel or pack;
one or more internal sleeve pockets having a third opening, disposed in the back side of the right front end panel for a panel or pack
a plurality of compression straps having a fastener at one or more ends of the compression straps, the plurality of compression straps are detachably attached in the back side of the size adjustable flexible abdominal belt; and
a plurality of anterior rigid panels slidably attached in the internal pocket and the one or more internal sleeve pockets.

2. The device as claimed in claim 1, wherein the plurality of compression straps include multiple elastic straps attached together to a strap support to form an elastic diamond shape.

3. The device as claimed in claim 1, wherein the connector includes a velcro attachment.

4. The device as claimed in claim 1, wherein the plurality of compression straps include memory retention medical grade elastic straps.

5. The device as claimed in claim 1, wherein the anterior rigid panels and the posterior rigid panels includes rectangular anatomical structures with a plurality of holes and/or perforations.

6. The device as claimed in claim 1, wherein the device further includes one or more of therapeutic gel packs slidably inserted into the internal pocket.

7. The device as claimed in claim 1, wherein the device further includes one or more of therapeutic gel packs slidably inserted to the one or more internal sleeve pockets.

8. The device as claimed in claim 1, wherein the one or more of therapeutic gel packs slidably attached to the one or more external sleeve pockets.

9. A device comprising:
a size adjustable flexible abdominal lower lumbar band including:
a back panel,
a right side contouring panel attached to the right side of the back panel,
a left side contouring panel attached to the left side of the back panel,
a right front end panel attached to one end of the right side contouring panel, and
a left front end panel attached to one end of the left side contouring panel;
a connector for connecting a first end and a second end of the size adjustable flexible abdominal belt;
an internal pocket disposed in a mid-portion of a front side of the size adjustable flexible abdominal belt, the internal pocket having a first opening for accessing the internal pocket;
one or more internal sleeve pockets having a third opening, disposed in the front side of the right front end panel and the left front end panel;
an external pocket disposed in a mid-portion of a back side of the size adjustable flexible abdominal belt, the external pocket having a second opening for accessing the external pocket;
one or more external sleeve pockets having a fourth opening, disposed in the back side of the right front end panel and the left front end panel;
a plurality of compression straps having a fastener at one or more ends of the compression straps, the plurality of compression straps are detachably attached in the back side of the size adjustable flexible abdominal belt, wherein the plurality of compression straps include multiple elastic straps attached together to a strap support to form an elastic diamond shape;
a plurality of anterior rigid panels detachably attached in the one or more internal sleeve pockets;
a plurality of posterior rigid panels detachably attached in the one or more external sleeve pockets; and
one or more therapeutic gel packs detachably attached to the internal pocket.

10. The device as claimed in claim 9, wherein the connector includes a velcro attachment.

11. The device as claimed in claim 9, wherein the plurality of compression straps include memory retention medical grade elastic straps.

12. The device as claimed in claim 9, wherein the anterior rigid panels and the posterior rigid panels include rectangular structures with a plurality of holes.

13. The device as claimed in claim 9, wherein the device further includes one or more of therapeutic gel packs detachably attached to the one or more internal sleeve pockets.

14. The device as claimed in claim 13, wherein the one or more of therapeutic gel packs detachably attached to the one or more external sleeve pockets.

15. A device comprising:
a size adjustable flexible abdominal belt including:
a back panel,
a right side contouring panel attached to the right side of the back panel,
a left side contouring panel attached to the left side of the back panel,
a right front end panel attached to one end of the right side contouring panel, and
a left front end panel attached to one end of the left side contouring panel;
a connector for connecting a first end and a second end of the size adjustable flexible abdominal belt;
an internal pocket disposed in a mid-portion of a front side of the size adjustable flexible abdominal belt, the internal pocket having a first opening for accessing the internal pocket;
an external pocket disposed in a mid-portion of a back side of the size adjustable flexible abdominal belt, the external pocket having a second opening for accessing the external pocket;
one or more internal sleeve pockets having a third opening, disposed in the front side of the left front end panel;
a plurality of compression straps having a fastener at one or more ends of the compression straps, the plurality of compression straps are detachably attached in the back side of the size adjustable flexible abdominal belt, the detachable compression straps are made from urethane; and
a plurality of anterior rigid panels detachably attached in the internal pocket and the one or more internal sleeve pockets.

16. The device as claimed in claim 15, wherein the connector includes a velcro attachment.

17. The device as claimed in claim 15, wherein the plurality of compression straps include memory retention medical grade elastic straps.

18. The device as claimed in claim 15, wherein the anterior rigid panels and the posterior rigid panels include rectangular structures with a plurality of holes.

19. The device as claimed in claim 15, wherein the device further includes one or more of therapeutic gel packs detachably attached to the one or more internal sleeve pockets.

20. The device as claimed in claim 19, wherein the one or more of therapeutic gel packs detachably attached to the one or more external sleeve pockets.
